(19) United States
(12) Patent Application Publication
(10) Pub. No.: US 2006/0258939 A1
(43) Pub. Date: Nov. 16, 2006

(54) Wearable glucometer

(75) Inventors: Benny Pesach, Rosh-Ha'Ayin (IL); Ron Nagar, Tel-Aviv (IL); Shai Ashkenazi, Rehovot (IL); Gidon Pesach, Tel-Aviv, IL (US); Avner Adoram, Ramat-Hasharon (IL)

Correspondence Address: WOLF, BLOCK, SCHORR & SOLIS-COHEN LLP, 250 PARK AVENUE, NEW YORK, NY, 10177, US

(73) Assignee: Glucon, Inc., Boulder, CO (US)

(21) Appl. No.: 10/559,974

(22) PCT Filed: Jun. 8, 2004
(86) PCT No.: PCT/IL04/00483

§ 371 Date: Jun. 29, 2006

Related U.S. Application Data
(60) Provisional application No. 60/476,623, filed on Jun. 9, 2003.

(51) Int. Cl. A61B 8/00

(52) U.S. Cl. 600/438

(57) ABSTRACT

Apparatus for assaying an analyte of blood in a patient's blood vessel comprising: a mounting module adapted so that it can be adhered to the skin of the patient overlying a tissue region comprising the blood vessel; a sensor unit mounted to the module that generates signals responsive to characteristics of the tissue region; and a controller that receives the signals and uses received signals to assay the analyte and to determine a degree to which the sensor unit is aligned with the blood vessel.

### FIELD OF THE INVENTION

The invention relates to wearable apparatus that can be coupled to a body and continuously assay a substance in the body for an extended period of time and in particular wearable apparatus for continuously monitoring glucose levels in a body.

### BACKGROUND OF THE INVENTION

Methods and apparatus for determining blood glucose levels for use in the home, for example by a diabetic who must monitor blood glucose levels frequently, are available. These methods and associated devices are generally invasive and usually involve taking blood samples by finger pricking. Often a diabetic must determine blood glucose levels many times daily and finger pricking is perceived as inconvenient and unpleasant. To avoid finger pricking diabetics tend to monitor their glucose levels less frequently than is advisable.

Non-invasive in-vivo methods and apparatus for monitoring blood glucose are known. PCT Publication WO 98/38904, the disclosure of which is incorporated herein by reference, describes a “non-invasive, in-vivo glucometer” that uses a photoacoustic effect to measure a person's blood glucose. PCT Publication WO 02/15776, the disclosure of which is incorporated herein by reference, describes locating a blood vessel in the body and determining glucose concentration in a bolus of blood in the blood vessel. The glucose concentration in the blood bolus is determined by illuminating the bolus with light that is absorbed and/or scattered by glucose to generate photoacoustic waves in the bolus. Intensity of the photoacoustic waves, which is a function of glucose concentration, is sensed and used to assay glucose in the bolus. Apparatus for determining glucose levels is hereinafter referred to as a “glucometer”.

Wearable glucometers are known, are generally based on near-infrared (NIR) spectroscopic methods and usually comprise a light source and optical detector that are attached to the patient's finger, wrist or other part of the body. Wearable NIR glucometers are described in U.S. Pat. No. 6,241,663 to Wu, et al. and U.S. Pat. No. 5,551,422, to Simonsen et al., the disclosures of which are incorporated herein by reference.

### SUMMARY OF THE INVENTION

An aspect of some embodiments of the present invention relates to providing a wearable glucometer that may be mounted to a patient's skin in alignment with a blood vessel in the patient's body and thereafter repeatedly assay glucose in blood in the blood vessel for a relatively extended period of time.

It is generally advantageous to determine glucose levels for a patient from blood glucose levels. Prior art wearable. glucometers generally do not distinguish between glucose levels in blood and glucose levels in interstitial fluid and cannot therefore assure that glucose assays they provide are blood glucose levels. Unlike prior art glucometers, a glucometer in accordance with an embodiment of the invention provides measurements of glucose levels that are substantially unadulterated by glucose levels in interstitial fluid.

An aspect of some embodiments of the present invention relates to providing a glucometer comprising apparatus useable to align the glucometer with the blood vessel.

An aspect of some embodiments of the present invention relates to providing a glucometer comprising imaging apparatus useable to image features below the skin of the patient. Images of features below the skin provided by the imaging apparatus are optionally used to align the glucometer with the blood vessel.

In some embodiments of the present invention, the glucometer comprises at least one acoustic transducer and a controller that controls the at least one transducer to acoustically image features in the patient's body beneath the skin. Optionally, the controller displays an acoustic image of the features and/or generates a signal responsive to the acoustic image that enables the glucometer to be aligned with the blood vessel.

In some embodiments of the present invention, the glucometer comprises a light source, at least one acoustic transducer and a controller. The light source is controlled to illuminate tissue beneath the skin with light that stimulates photoacoustic waves in the tissue. Signals generated by the at least one acoustic transducer responsive to the photoacoustic waves are transmitted to the controller. The controller processes the signals to generate a photoacoustic image of features below the skin. Optionally, the controller displays the image and/or generates a signal responsive to the photoacoustic image that enables the glucometer to be aligned with the blood vessel.

Following alignment with the blood vessel the glucometer is fixed in place on the patient's skin. Thereafter, the glucometer repeatedly assays glucose in a blood bolus in the blood vessel using photoacoustic methods known in the art such as those described in PCT publication WO 02/15776, the disclosure of which is incorporated herein by reference. However, in time, a glucometer initially aligned with a blood vessel may become misaligned because of drift in the glucometer position on the skin or because of motion of the skin relative to the blood vessel.

An aspect of some embodiments of the present invention relates to providing a glucometer that can determine if it becomes misaligned with the blood vessel.

In some embodiments of the present invention, the glucometer determines that it is misaligned with the blood vessel responsive to a change in assay signals that it receives that cannot be explained by normal changes in blood glucose. For example, the glucometer might determine that amplitude or waveform changes in the signals or a relatively abrupt change in blood glucose level is a result of the glucometer becoming misaligned with the blood vessel and not a result of a change in blood glucose. For a glucometer, in accordance with an embodiment of the invention that can image features below the skin on which it is mounted, such as by using ultrasound or the photoacoustic effect, the glucometer periodically images the features. From the images, the controller determines if the glucometer is or is not aligned with the blood vessel.

An aspect of some embodiments of the present invention relates to providing a glucometer that is self-aligning.

In accordance with an embodiment of the invention the glucometer comprises at least one motor coupled to a component of the glucometer which the motor moves to realign the glucometer if the glucometer determines that it has become misaligned.

According to an embodiment of the invention the glucometer controls the motor to repeatedly impart motion to the component of the glucometer so that the glucometer continuously “wanders” over a region of the skin on which it is mounted. As it wanders the glucometer determines when it becomes aligned with a blood vessel. When it becomes aligned it performs an assay of glucose in the blood vessel.

The miventors have noted that apparatus, such as for example a glucometer in accordance with an embodiment of the invention, when mounted on the skin of a patient may exert an excessive amount of pressure on blood vessels beneath the skin. The pressure may be such that it causes a blood vessel located beneath the skin to collapse or deform to such an extent that blood flow is adversely affected.

An aspect of some embodiments of the present invention relates to providing a glucometer, which when mounted on the skin of a patient, if it exerts pressure to collapse or substantially deform a blood vessel beneath the skin reduces the pressure it exerts on the skin.

There is therefore provided in accordance with an embodiment of the present invention, apparatus for assaying an analyte of blood in a patient's blood vessel comprising: a mounting module adapted so that it can be adhered to the skin of the patient overlying a tissue region comprising the blood vessel; a sensor unit mounted to the module that generates signals responsive to characteristics of the tissue region; and a controller that receives the signals and uses received signals to assay the analyte and to determine a degree to which the sensor unit is aligned with the blood vessel.

Optionally, the sensor unit comprises at least one light source that illuminates the region with light at at least one wavelength that is absorbed and/or scattered by the analyte and generates photoacoustic waves in the region and at least one acoustic transducer that generates at least some of the signals responsive to the photoacoustic waves.

Optionally, the controller uses signals responsive to the photoacoustic waves to assay the analyte.

Additionally or alternatively, the light source illuminates the region with light that is absorbed by red blood cells.

In some embodiments of the present invention, the controller uses at least one characteristic of the photoacoustic signals to determine a degree to which the sensor unit is aligned with the blood vessel. Optionally, the at least one characteristic comprises a magnitude of the amplitude of the signal. Additionally or alternatively the at least one characteristic comprises shape of the signal. In some embodiments of the present invention, the at least one characteristic comprises a time dependence of the signal. In some embodiments of the present invention, the at least one characteristic comprises a power spectrum of the signal.

In some embodiments of the present invention, the controller controls the at least one transducer to transmit ultrasound into the region and wherein the at least one transducer generates at least some of the signals, responsive to ultrasound reflected from features comprised in the region. Optionally, the controller uses signals generated responsive to reflected ultrasound to determine a degree to which the sensor is aligned with the blood vessel.

In some embodiments of the present invention, the apparatus comprises a display screen. Optionally, the controller displays data that relates to a degree to which the sensor unit is aligned with the blood vessel on the display screen. Additionally or alternatively, the controller uses signals from the at least one acoustic transducer to generate an image of the blood vessel and displays the image on the screen.

In some embodiments of the present invention, the controller uses signals generated responsive to reflected ultrasound to generate an image of the blood vessel and displays the image on the screen. Additionally or alternatively the controller displays a fiducial on the screen and wherein a distance on the screen between the fiducial and the image of the blood vessel indicates a degree to which the sensor unit is misaligned with the blood vessel.

In some embodiments of the present invention, at least a portion of the sensor unit presses against the skin to provide optical and/or acoustic coupling of the sensor to the skin. Optionally, the controller uses signals received from the sensor unit to determine if the sensor portion exerts excessive pressure on the blood vessel.

There is further provided in accordance with an embodiment of the invention, apparatus for assaying an analyte of blood in a patient's blood vessel comprising: a mounting module adapted so that it can be adhered to the skin of the patient overlying a tissue region comprising the blood vessel; a sensor unit mounted to the module that generates signals responsive to characteristics of the tissue region wherein at least a portion of the sensor unit presses against the skin to provide optical and/or acoustic coupling of the sensor to the skin; a controller that receives the signals and uses received signals to assay the analyte and to determine if the sensor portion exerts excessive pressure on the blood vessel.

Optionally, the sensor unit comprises at least one light source that illuminates the region with light at at least one wavelength that generates photoacoustic waves in the region and at least one acoustic transducer that generates at least some of the signals responsive to the photoacoustic waves. Optionally, the controller controls the at least one transducer to transmit ultrasound into the region and wherein the at least one transducer generates at least some of the signals responsive to ultrasound reflected from features comprised in the region.

In some embodiments of the present invention, the controller uses the signals to generate an image of the blood vessel and if the image indicates that the blood vessel is deformed relative to a normative blood vessel shape, the controller determines that the sensor portion exerts excessive pressure.

In some embodiments of the present invention, the sensor portion position is movable relative to the mounting module in a direction substantially perpendicular to the skin so as to adjust pressure that the sensor unit portion exerts on the skin and thereby on the blood vessel.

Optionally, the position of the sensor portion is manually adjustable. Alternatively or additionally, the apparatus comprises a motor controllable to adjust the position of the sensor unit portion. Optionally, the controller controls the motor to adjust position of the sensor unit portion if the controller determines that the sensor unit portion exerts excessive pressure on the skin.

In some embodiments of the present invention, the mounting module comprises a frame having sides, which at least partially surround a region that receives the sensor unit. Optionally, the region that receives the sensor unit is an open region and when the sensor unit is positioned in the region no portion of the mounting module intervenes between the sensor unit and the skin. Optionally, the apparatus comprises an adhesive that attaches the sensor unit to the skin when the sensor unit is mounted in the open receiving region. Optionally, the adhesive is substantially transparent to light provided by the at least one light source. Additionally or alternatively, the adhesive is a relatively good conductor of sound and reduces acoustic impedance mismatch between the sensor unit and the skin.

In some embodiments of the present invention, the apparatus comprises a gel that optically and acoustically couples the sensor unit to the skin when the sensor unit is mounted in the open receiving region.

In some embodiments of the present invention, the frame comprises a panel that connects the sides and which intervenes between the sensor unit and the skin when the sensor unit is mounted in the receiving region. Optionally, the panel is flexible. Additionally or alternatively, the panel is substantially transparent to light provided by the at least one light source. Optionally, the panel is a relatively good conductor of sound.

In some embodiments of the present invention, the panel comprises an adhesive layer that bonds the panel and thereby the mounting module to the skin. Optionally, the adhesive is substantially transparent to light provided by the at least one light source. Optionally, the adhesive is a relatively good conductor of sound and reduces acoustic impedance mismatch between the panel and the skin.

In some embodiments of the present invention, the apparatus comprises a gel or oil that optically and acoustically couples the sensor unit to the panel when the sensor unit is mounted in the receiving region.

In some embodiments of the present invention, the frame comprises at least one elastic element that exerts a resilient force on the sensor unit substantially parallel to the plane of the frame to maintain the sensor unit securely in position in the frame. Optionally, the apparatus comprises at least one set screw having a position that limits motion of the sensor unit in a direction that the resilient force operates to move the sensor unit. Optionally, the set screw is mounted in the frame. Additionally or alternatively, the position of the set screw is adjustable manually.

In some embodiments of the present invention, the apparatus comprises a motor controllable to adjust the position of the set-screw. Optionally, the controller controls the motor to adjust position of the sensor unit if signals received from the sensor unit indicate alignment of the sensor unit is unsatisfactory.

In some embodiments of the present invention, the apparatus comprises a motor controllable to adjust the position of the sensor unit relative to the mounting module in a direction parallel to the skin and wherein the controller controls the motor to adjust position of the sensor unit if signals received from the sensor unit indicate alignment of the sensor unit is unsatisfactory.

In some embodiments of the present invention, the analyte is glucose.

### BRIEF DESCRIPTION OF FIGURES

Non-limiting examples of embodiments of the present invention are described below with reference to figures attached hereto, which are listed following this paragraph. In the figures, identical structures, elements or parts that appear in more than one figure are generally labeled with a same numeral in all the figures in which they appear. Dimensions of components and features shown in the figures are chosen for convenience and clarity of presentation and are not necessarily shown to scale.

FIG. 1A schematically shows an exploded view of a glucometer comprising a mounting module, a sensor pack, an actuator pack and an interface module, in accordance with an embodiment of the present invention;

FIG. 1B schematically shows the sensor pack shown in FIG. 1A mounted to the mounting module, in accordance with an embodiment of the present invention;

FIG. 1C schematically shows the actuator pack mounted to the sensor pack and mounting module shown in FIGS. 1A and 1B, in accordance with an embodiment of the present invention;

FIG. 1D schematically shows the glucometer shown in FIG. 1A in an exploded view completely assembled, in accordance with an embodiment of the present invention;

FIG. 1E schematically shows a cross section view of the assembled glucometer shown in FIG. 1D, in accordance with an embodiment of the present invention;

FIGS. 2A-2E schematically illustrate mounting the glucometer shown in FIG. 1E to the skin of a patient's body to assay glucose in a blood vessel located in a region beneath the skin, in accordance with an embodiment of the present invention;

FIG. 3A schematically shows a mounting module for another glucometer, in accordance with an embodiment of the present invention;

FIG. 3B schematically shows a sensor pack adapted for use with the mounting module shown in FIG. 3A, in accordance with an embodiment of the present invention;

FIG. 3C schematically shows the sensor pack shown in FIG. 3B being mounted to the mounting module shown in FIG. 3A, in accordance with an embodiment of the present invention;

FIG. 3D schematically shows the sensor pack in FIG. 3B mounted in the mounting module shown in FIG. 3A, in accordance with an embodiment of the present invention; and

FIG. 4 schematically shows a cross section view of another glucometer mounted to a region of a patient's skin, in accordance with an embodiment of the present invention.

### DETAILED DESCRIPTION OF EXEMPLARY EMBODIMENTS

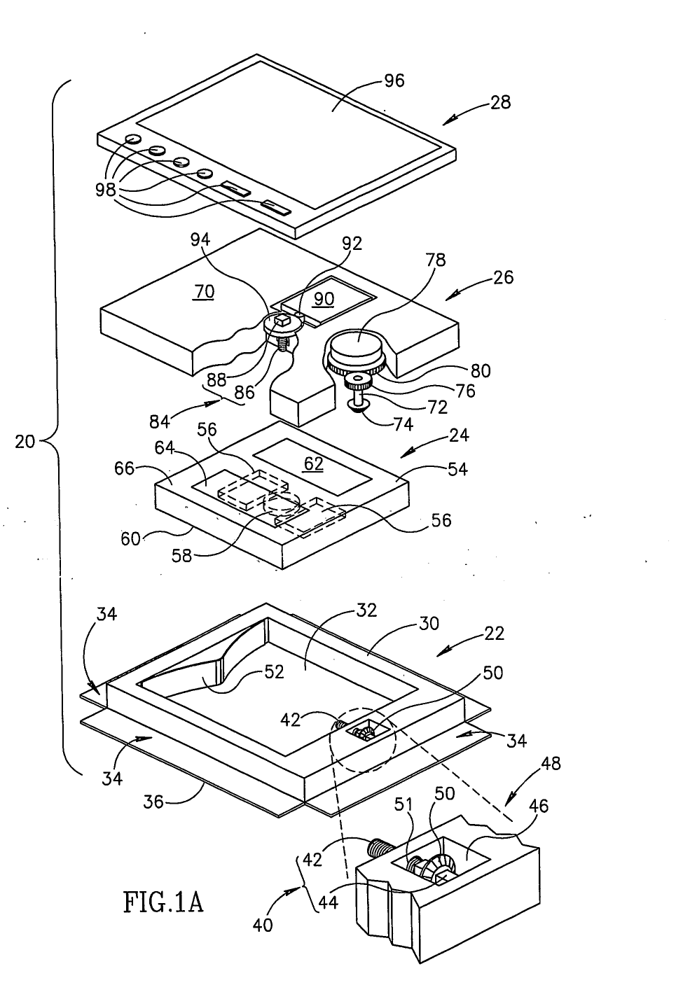

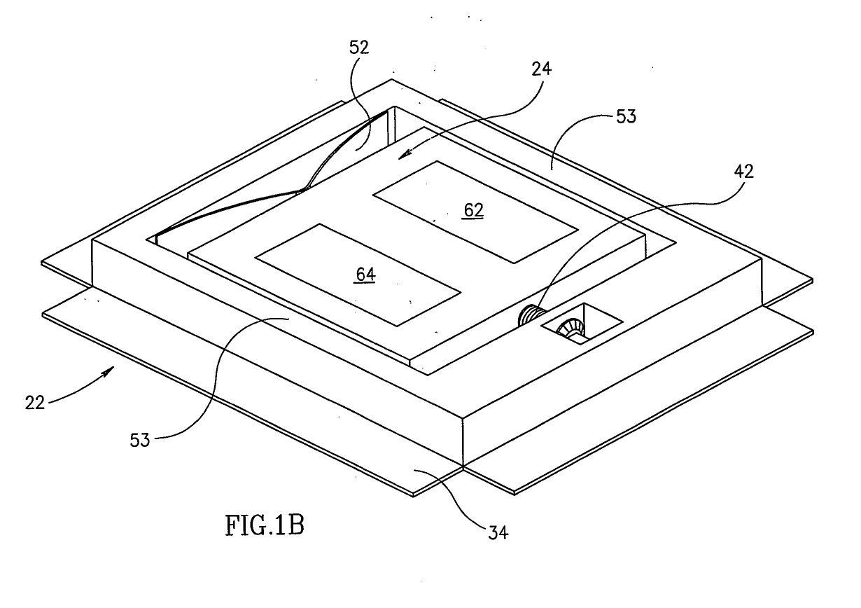

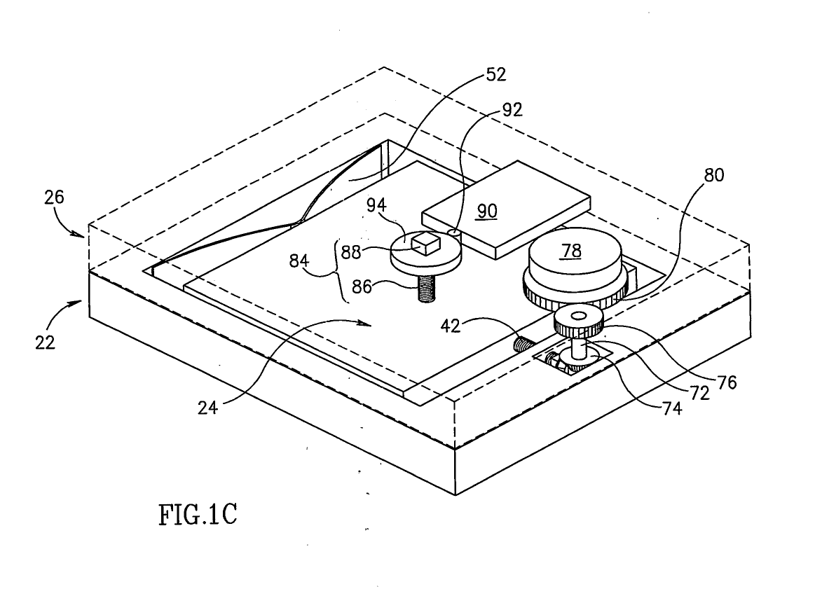

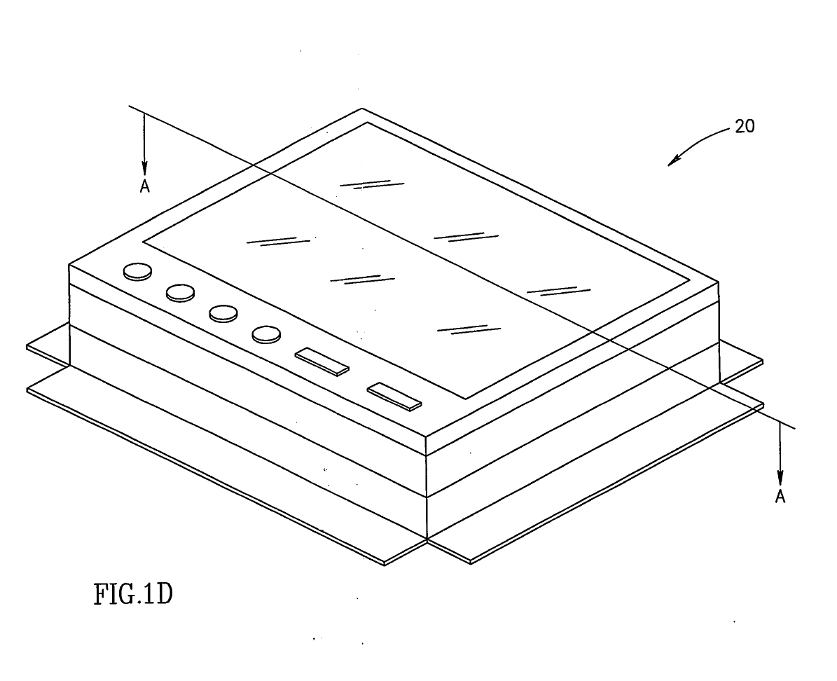

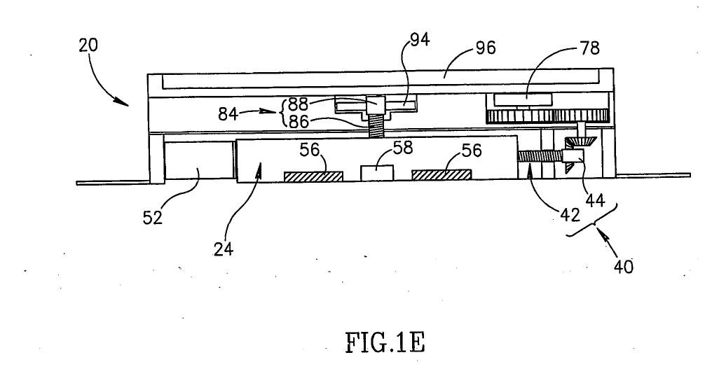

FIG. 1A schematically shows an exploded view of a wearable glucometer 20, in accordance with an embodiment of the present invention. Glucometer 20 optionally comprises a mounting module 22, a sensor pack 24, an actuator pack 26 and an interface module 28.

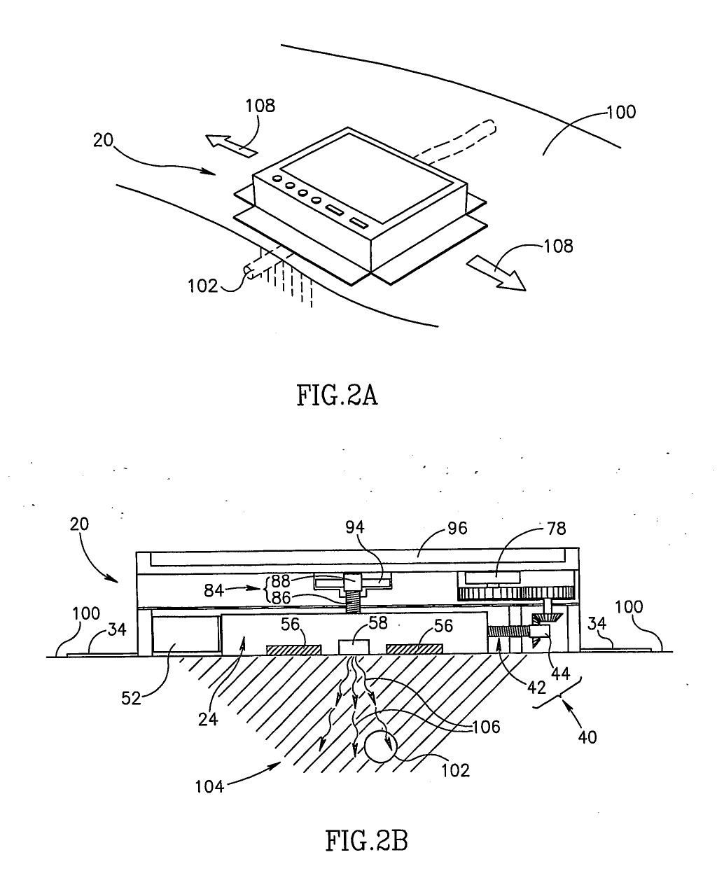

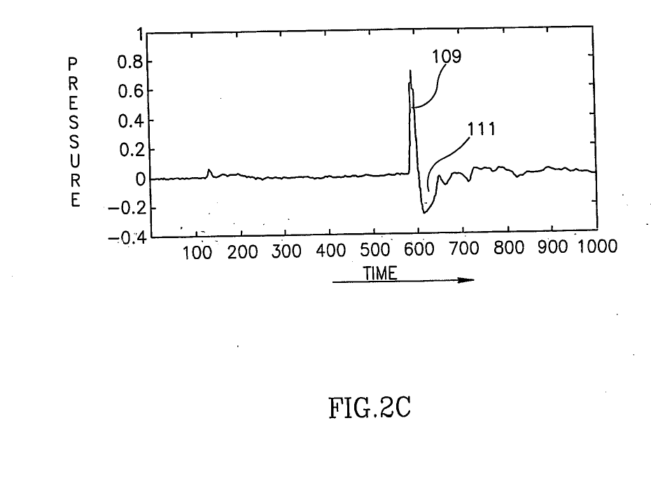

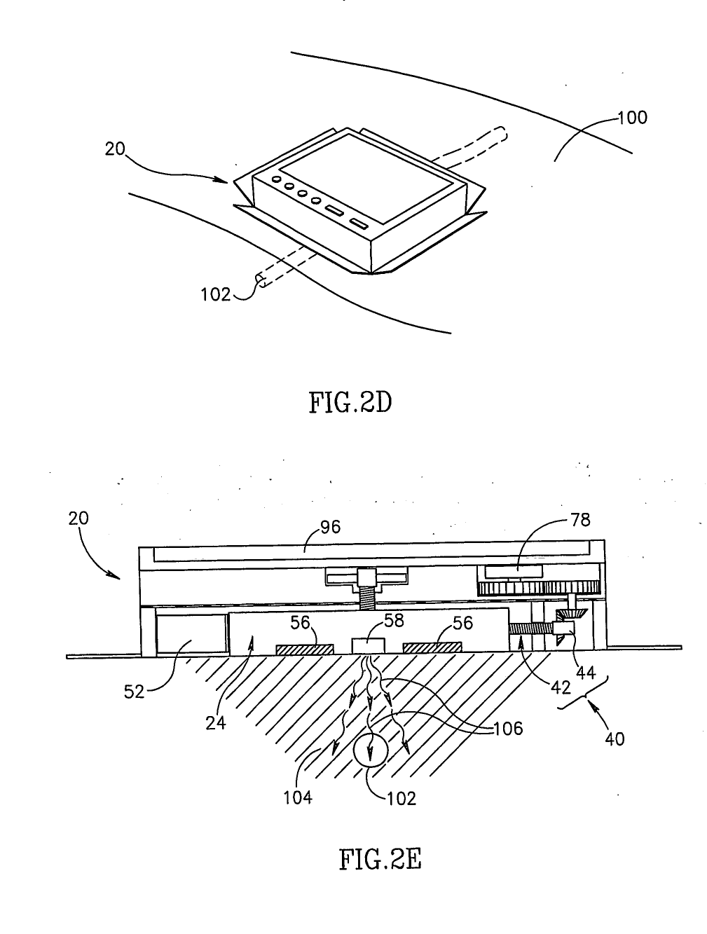

Mounting module 22 optionally comprises a frame 30 surrounding an open region 32. Frame 30 is attached to at least one flexible skirt 34 having an underside 36 covered with an adhesive layer (not shown) suitable for adhering the skirt to a region of a patient's body. By way of example mounting module 22 comprises four skirts 36. The adhesive is such that it may be used to attach mounting module 22 for a relatively long period of time to a region of a patient's body. The adhesive layer is optionally covered with a protective film (not shown) which is removed when the layer is to be used. A procedure for mounting mounting module 22 and glucometer 20 to a patient is discussed in the description of FIGS. 2A-2F.

Optionally, a set screw 40 comprising a threaded shaft 42 and an optionally square shaft 44 is mounted in frame 30. Features of set screw 40 are shown in a magnified view for convenience of presentation in an inset 48. Threaded shaft 42 is screwed into a threaded hole 51 in frame 30 and a portion of the threaded shaft generally protrudes into open region 32. A depth to which threaded shaft 42 is screwed into the threaded hole in frame 30 determines an amount by which the threaded portion protrudes into open region 30. Square shaft 44 and generally a portion of threaded shaft 42 protrude into a hole 46 formed in frame 30.

Optionally a “transmission” pinion gear 50 is slidingly mounted to square shaft 44 so that the shaft and gear rotate together but the shaft is free to move relative to the pinion gear in a direction along the length of the shaft. Mounting of transmission pinion gear 50 to shaft 44 so that the pinion gear and shaft turn together and move relative to each other is accomplished by forming the pinion gear with a square hole having dimensions slightly larger than dimensions of the cross section of square shaft 44. Transmission pinion gear 50 is then slidingly mounted to shaft 44 by positioning the shaft in the hole.

Optionally, position washers or sleeves (not shown) formed from a suitable material are mounted to set screw 40 on either side of pinion gear 50 to maintain the pinion gear in a fixed position in hole 46. An “opposition” spring 52, which may be any suitable resilient material such as a resiliently compressible material, at least one coil spring, or as is shown in FIG. 1A, optionally a leaf spring, is mounted on frame 30 opposite set screw 40. Functioning of the opposition spring 52 and set screw 40 is described below.

Sensor pack 24 optionally comprises a support plate 54. At least one acoustic transducer 56 and at least one light source 58 are mounted on a bottom side 60 of support plate 54. At least one transducer 56 may comprise any of various configurations of transducers known in the art and may for example be a single transducer or a plurality of transducers operating optionally as a phased array. By way of example, two acoustic transducers 56 and one light source 58 are mounted in support plate 54. The acoustic transducers and light source are not normally seen in the perspective of FIG. 1 and are shown in ghost lines.

Optionally, a controller 62 and a power source 64 are mounted on a top side 66 of mounting module 22. Control and power connections between controller 62, power source 64, light source 58 and acoustic transducers 56 are provided using any of various devices and methods known in the art.

Sensor pack 24 has a shape and dimensions that enable the sensor pack to be placed in open region 32 of mounting module 22 between opposition spring 52 and set screw 40. Opposition spring 52 operates to resiliently press sensor pack 24 against set screw 40. An amount by which set screw 40 protrudes into open region 32 determines a position of sensor pack 24 relative to frame 30 in a direction along which set screw 40 protrudes into open region 32. FIG. 1B schematically shows sensor pack 24 positioned inside open region 32 of mounting module 22 with opposition spring 52 pressing the sensor pack against set screw 40. Optionally, the position of sensor pack 24 relative to sides 53 of frame 30 is not adjustable and the dimensions of the sensor pack are such that it fits snugly between sides 53 of the frame.

Actuator pack 26 optionally comprises an actuator pack support plate 70 that is mountable to mounting module 22, using any of various methods and techniques in the art, so that the actuator pack is aligned with open region 32 and therefore with sensor pack 24 positioned in the open region. Optionally, actuator pack 26 is mounted to mounting module 22 using appropriate latching elements that enable the actuator pack to be repeatedly mounted and easily detached from the mounting module.

Actuator support plate 70 has a shaft 72 mounted thereto, which is fit with a drive pinion gear 74 and a drive gear 76. An optionally electromagnetic motor 78 having a motor gear 80 is mounted in mounting module 22 so that the motor gear meshes with drive gear 76. Support plate 70 is partially cut-away to show details of motor 78, shaft 72 and associated components.

When actuator pack 26 is mounted to mounting module 22 drive pinion gear 74 meshes with transmission pinion gear 50 comprised in mounting module 22. Motor 78 is controllable by controller 62 to rotate drive gear 72 and thereby, depending upon a direction in which motor 78 rotates the drive gear, by how much set screw 40 protrudes into open region 32. FIG. 1C schematically shows actuator pack 26 mounted to mounting module 22. External outlines of actuator pack 26 and mounting module 22 are shown in dashed lines and internal features normally hidden in the view of FIG. 1C and germane to the discussion are shown in solid lines.

A pressure screw 84 comprising a threaded shaft 86 and a drive shaft 88 optionally having a square cross section is optionally screwed into a threaded hole (not shown) in support plate 70. Actuator support plate 70 is partially cutaway in FIG. 1A to show details of pressure screw 84. Pressure screw 84 is optionally rotated by a piezoelectric motor 90, such as for example a piezoelectric motor of a type shown in U.S. Pat. No. 5,616,980 or a multilayer piezoelectric motor such as described in PCT Publication WO 00/74153. Piezoelectric motor 90 optionally comprises a friction nub 92 and is resiliently urged to press the friction nub against a “friction” driving wheel 94 by a suitable spring or resilient body (not shown) using any of various methods and devices known in the art.

Friction wheel 94 is slidingly mounted to drive shaft 88 so that the friction wheel and driving shaft rotate together but the drive shaft is free to move relative to the friction wheel in a direction along the drive shaft. Friction wheel 94 is optionally mounted to drive shaft 88 similarly to the manner in which transmission pinion gear 50 is slidingly mounted to set screw 40. Vibrations in piezoelectric motor 90 generate vibrations in friction nub 92 that rotate friction wheel 94 to rotate pressure screw 86 in a desired direction to raise or lower the pressure screw relative to actuator pack mounting module 22. Because friction wheel is *slidingly mounted to drive shaft 88 the friction wheel does not move up or down as pressure screw moves up and down. When actuator pack 26 is mounted to mounting module 22, as set screw 40 is rotated to lower pressure screw 84, the pressure screw presses on sensor pack 24 and exerts a force on the sensor pack that tends to move the sensor pack downward relative to the mounting plate.

In some embodiments of the present invention rather than press on sensor pack 24, pressure screw 84 presses on an elastic element sandwiched between the pressure screw and the sensor pack. When the pressure screw is rotated to move the pressure screw towards or away from sensor pack 24, the elastic element compresses or expands respectively. An amount of pressure that the elastic element exerts on sensor pack 24 increases as the elastic body compresses and decreases as the elastic body expands.

Interface module 28 optionally comprises a display screen 96 and appropriate buttons 98 for transmitting commands to controller 62. Interface module 28 may be an integral part of actuator pack 26 or may be mountable to the actuator pack using any of various devices and techniques known in the art. In some embodiments of the invention, interface module 28 comprises circuitry for wireless transmission of data to for example a display screen, control console, computer or data storage device that is not incorporated in glucometer 20. For example, interface module might be equipped to relay data and accept control signal from a desktop computer display or to relay data to a data storage device worn on another part of the body.

FIG. 1D schematically shows glucometer 20 shown in FIG. 1A in an exploded view completely assembled. FIG. 1E schematically shows a cross section view of assembled glucometer 20 along the plane indicated by line AA shown in FIG. 1D. Optionally, interface module 28, actuator pack 26 and sensor pack 24 are connected to each other so that may be removed from mounting module 22 as a single unit. It is noted that whereas the connection between interface module 28 and actuator pack 26 may be relatively rigid, the connection between the actuator pack and sensor pack 24 is such that it enables motion of the sensor pack relative to the actuator pack in a direction parallel to the axis of pressure screw 84. Actuator pack 26 may be connected to mounting module 22 by any of many different suitable connectors known in the art so that the actuator pack, and sensor pack and interface module connected to the actuator pack, may be easily connected and disconnected from the mounting module.

FIGS. 2A-2F schematically illustrate mounting glucometer 20 to the skin 100 of a patient's body to assay glucose in a blood vessel 162 located in a region 104 beneath the skin.

To prepare for the process of attaching glucometer 20 to skin 100, a control signal is input to the glucometer via interface buttons 98 instructing controller 62 (FIGS. 1A and 1B) to operate in a “manual positioning mode”. In the manual positioning mode controller 62 controls motor 78 (FIGS. 2B, 1A and 1C) to adjust position of set screw 40 so that it protrudes into open region 32 by an amount substantially equal to half of its maximum protrusion into the open region. After adjusting position of set screw 40, optionally, controller 62 does not make any further adjustment to the position of set screw 40 while in the manual positioning mode. In addition, controller 62 controls piezoelectric motor 90 to position pressure screw 84 so that it extends downward in the direction of sensor pack 24 by an amount substantially equal to half of its maximum downward extension. A coupling gel that transmits acoustic waves as well as light at wavelengths provided by light source 58 is optionally applied to sensor pack 24 so that it covers light source 58 and transducers 56.

As shown in a perspective partially cutaway view in FIG. 2A and in a cross section view in FIG. 2B, glucometer 20 is then positioned on skin 100 at a location for which it is expected that light source 58 will be located substantially over blood vessel 102. In addition, glucometer 20 is oriented so that the axis of set screw 40 (FIG. 2B) is substantially perpendicular to the length of the blood vessel. When positioned on line 100, controller 62 controls light source 58 (FIG. 1A) to illuminate region 104 with light, represented by wavy arrows 106 in FIG. 2B that is preferentially absorbed by blood, such as light at a wavelength relatively strongly absorbed by hemoglobin. While light 106 illuminates region 104, the patient or a person aiding the patient moves glucometer 20 back and forth on skin 100, preferably in a direction that is substantially perpendicular to a direction along which blood vessel 102 extends. Direction of back and forth motion of glucometer 20 is indicated by direction of block arrows 108.

Light 106 generates photoacoustic waves in region 104, which are received by acoustic transducers 56. Signals generated by the acoustic sensors responsive to the received photoacoustic waves are transmitted to controller 62. Controller 62 processes the signals to determine when light source 58 is substantially located directly over blood vessel 102. Optionally, controller 62 determines that light source 58 is directly over blood vessel 102 when signals generated by transducers 56 indicate that photoacoustic waves reaching both transducers and exhibiting characteristics typical of signals generated by the presence of a blood vessel having a same intensity. Optionally, controller 62 determines that light source 58 is aligned with blood vessel 102 when intensity of photoacoustic waves reaching transducers 56 and exhibiting characteristics, such as for example of signal waveform or power spectrum, typical of signals generated by the presence of a blood vessel reach a maximum.

FIG. 2C is a schematic graph of pressure sensed by transducers 56 as a function of time that results from photoacoustic waves stimulated by light 106 when light source 58 is substantially aligned with blood vessel 102. Sensed pressure is measured along the abscissa in arbitrary units and time along the abscissa is measured in microseconds. The graph shows a large well-defined peak 109 followed by a “trough” 111 that are typical of photoacoustic waves stimulated in a blood vessel by light absorbed by blood. As alignment of light source 58 with blood vessel 102 deteriorates shape of peak 109 and trough 111 changes and departs from the typical shape shown in the graph and/or amplitude of the peak and/or of the trough decrease.

When controller 62 determines that light source 58 is aligned with blood vessel 102 it generates a signal alerting the patient and/or a person aiding the patient that the glucometer is aligned. The patient or the patient's aid peels off the protective film covering the adhesive layer on skirts 34 and presses the skirts to skin 100 to adhere the skirt and thereby to attach glucometer 20 to the skin. FIG. 2D schematically shows skirt 34 lifted off skin 100 to enable the protective films to be peeled off the adhesive layers. FIG. 2E schematically shows glucometer 20 after skirts 34 are adhered to skin 100 and glucometer 20 securely in place on the skin and aligned with blood vessel 102.

In some embodiments of the invention, alignment of glucometer 20 with blood vessel 102 may be accomplished responsive to an image displayed on screen 96 of internal features of the patient's body that are located below a region of skin 100 on which the glucometer is positioned. For example, in embodiments of the present invention for which at least one transducer 56 comprises a phased array, controller 62 may generate the image using signals that the phased array provides responsive to photoacoustic waves in the region stimulated by light 106. Controller 62 may display the image on screen 96 together with a suitable fiducial such as for example a cross hair or a small circle. As glucometer 20 is moved over skin 100 and an image of blood vessel 102 is displayed on screen 96, the patient or a person aiding the patient aligns the image of the blood vessel with the fiducial to align the glucometer with the blood vessel.

Whereas glucometer 20 is described as using a photoacoustic effect to locate blood vessel 102 and align sensor pack 24 with the blood vessel, in some embodiments of the invention, a glucometer alternatively or additionally uses active ultrasound to locate the blood vessel and align the sensor pack. In an active ultrasound method, controller 62 controls ultrasound transducers 56 to transmit ultrasound into region 104. The controller uses reflections of the ultrasound from features in region 104 to locate blood vessel 102 and determine when sensor pack 24 is aligned with glucometer.

In some embodiments of the invention, the reflected ultrasound may be used to generate “active ultrasound” images of features below regions of skin 100 on which glucometer 20 is positioned, which images are displayed on screen 96. As in the case of images generated responsive to photoacoustic waves described above, an image of blood vessel 102 in the active ultrasound images may be aligned with a fiducial to align glucometer 20 with the blood vessel.

In some embodiments of the invention, a fiducial mark or marks, such as a tattoo or an artificial insert, which is aligned with blood vessel 102, may be placed on or under skin 100. Glucometer 20 may then be aligned with blood vessel 102 by aligning the glucometer with the fiducial mark or marks. In some embodiments of the invention, the fiducial mark, or marks, is configured so that it has a unique acoustic or photoacoustic signature. Glucometer 20 may then be aligned using active ultrasound or the photoacoustic effect. A suitable fiducial mark or marks may for example be made relative to a position of glucometer 20 when the glucometer is aligned with blood vessel 102 a first time. Thereafter, if glucometer 20 is removed from the skin 100 it can be replaced and positioned on skin 100 substantially aligned with blood vessel 102 by referencing the position of the glucometer to the fiducial mark or marks.

After attachment to skin 100, optionally, sensor pack 24 is removed from mounting module 22 and an adhesive for coupling light source 58 and acoustic transducers 56 to skin 100 for a relatively extended period is applied to the sensor pack. The adhesive is chosen so that it provides both acoustic and optical coupling of sensor pack to the skin and reduces optical and acoustic impedance mismatch between the skin and light source 58 and acoustic transducers 56 respectively. Optionally, the adhesive is sufficiently flexible to allow motion of the sensor pack relative to the skin but sufficiently viscous to prevent loss of the adhesive as a result of leakage of the adhesive from between the sensor pack and the skin. Optionally, the adhesive is such that the sensor pack may repeatedly be removed from and recoupled to skin 100 without having to replace the adhesive. The use of an adhesive for coupling sensor pack 24 to skin 100 for a relatively extended time period can be advantageous. Loss of an adhesive over an extended time period as a result of leakage of the adhesive from between the sensor pack and the skin will in general be substantially less than leakage of a coupling gel.

The inventors have determined that medical grade flexible adhesives, such as Bioflex RX268S marketed by Scapa Medical of Inglewood Calif., or a clear polyester adhesive ARcare 8890 marketed by Adhesive research Inc. from Limerick, Ireland, or a hydrogel adhesive described in U.S. Pat. No. 5,394,877, the disclosure of which is incorporated herein by reference, is suitable and optionally used for the practice of the present invention.

After application of the adhesive, sensor pack 24 is replaced into mounting module 22 for monitoring of the patient's glucose over a relatively extended time period and pressed against skin 100 to assure that the adhesive properly couples the sensor pack to the skin. Glucometer 20 is then controlled to operate in an assay mode. In the assay mode controller 62 controls light source 58 to illuminate region 104 with light at at least one wavelength that is scattered and or absorbed by glucose. Signals generated responsive to photoacoustic waves generated in blood in blood vessel 102 by the light is used to determine concentration of glucose in the blood. Any of various methods for assaying glucose in blood in blood vessel 102 responsive to a photoacoustic effect may be used to determine concentration of glucose in the blood. For example, photoacoustic assay methods described in PCT publication WO 02/15776 cited above or in U.S. provisional application 60/439,435 filed on Jan. 13, 2003, the disclosure of which is incorporated herein by reference are suitable for practice of the present invention.

In the assay mode, glucometer 20 monitors position of sensor pack 24 relative to blood vessel 102 to determine if the sensor pack maintains alignment with the blood vessel. In some embodiments of the invention, glucometer periodically monitors the relative position of sensor pack 24 to blood vessel 102 by periodically switching from the assay mode to an “automatic” positioning mode and back to the assay mode. In the automatic positioning mode glucometer 20 uses a photoacoustic effect and/or active ultrasound to determine if sensor pack 24 has become misaligned with blood vessel 102.

In some embodiments of the invention controller 62 compares photoacoustic or active ultrasound signals with “normative” photoacoustic or active ultrasound signals respectively, which are received when sensor pack 24 is aligned with blood vessel 102, to determine if the sensor pack has become misaligned. Normative signals may for example be signals that are recorded when glucometer 20 is known to be aligned with blood vessel 102 at a time immediately following a first manual alignment procedure. By way of another example, normative signals may also be standard signals generated responsive to experience and experimentation under various conditions of alignment of a glucometer, similar to glucometer 20 with blood vessels.

If controller 62 determines that sensor pack 24 is misaligned, controller 62 optionally controls motor 78 to adjust an amount by which set screw 40 protrudes into open region 32 of mounting module so as to align the sensor pack. It is noted that for situations in which an adhesive is used to couple glucometer 20 to skin 100, an amount by which set screw 40 can adjust position of sensor pack 24 to align the glucometer may be limited by how much the skin can be stretched before causing discomfort to the patient. Motion of sensor pack responsive to a change in position of set screw 40 may be limited by “skin stretch discomfort to a maximum displacement of between 0.5 mm and 1 mm. In some embodiments of the present invention, if controller 62 cannot adjust the position of set screw 40 so as to provide satisfactory alignment of sensor pack 24 with blood vessel 102 the controller generates an alarm to alert the patient that user intervention is required.

In some embodiments of the invention glucometer 20 determines that sensor pack 24 has become misaligned with blood vessel 102 responsive to a change in assay signals that it receives and/or a change in glucose assay that cannot be explained by normal changes in blood glucose. For example, changes in signal amplitude and/or wave form and/or power spectrum may alert glucometer 20 to sensor pack 24 becoming misaligned. Or for example, glucometer 20 might determine that a relatively abrupt change in blood glucose level is a result of sensor pack 24 becoming misaligned and not as a result of a change in blood sugar level. In such instances, glucometer 20 optionally enters an automatic adjustment mode and adjusts position of sensor module 24. Optionally, glucometer 20 does not enter into an automatic positioning mode in which it uses active ultrasound or a photoacoustic effect stimulated by light preferentially absorbed by blood to adjust position of sensor module 24 as described above. Instead glucometer adjusts position of sensor pack 24 responsive to the anomalous change and adjusts position of the sensor pack to minimize the anomaly. If the anomaly cannot be satisfactorily mitigated controller 62 optionally generates a suitable alarm indicating that user intervention is required.

It is noted that glucometer 20 has, by way of example, a single set screw 40 and that therefore the position of sensor pack 24 is adjustable along only one direction, the direction along which the screw moves into and out of open region 32. As noted above, glucometer 20 is positioned so that set screw 40 is substantially perpendicular to the length of blood vessel 102. As a result, alignment of sensor pack 24 is substantially insensitive to displacement perpendicular to set screw 40 and a single positioning screw is generally sufficient to maintain sensor pack 24 aligned with blood vessel 102. However, in some embodiments of the present invention a glucometer similar to glucometer 20 comprises two positioning screws oriented at right angles to each other and each driven by a suitable motor and transmission system. A glucometer having two orthogonal positioning screws can be advantageous, for example if it is difficult to orient a glucometer having a single set screw so that the set screw is substantially perpendicular to a length of a blood vessel.

In addition to monitoring alignment of sensor pack 24 with respect to blood vessel 102, glucometer 20 optionally controls pressure that sensor pack 24 exerts on region 104. In particular, in some embodiments glucometer 20 monitors the condition of blood vessel 102 to determine if pressure that sensor pack 24 exerts on region 104 changes the shape of the blood vessel and interferes with blood flow. For example, in some cases a pressure of about 8 mm Hg applied to a blood vessel, such as blood vessel 102, may result in collapse or, substantial changes in the shape of the blood vessel. In order to rectify instances in which sensor pack 24 overpressures region 104, when the glucometer operates in the automatic positioning mode it generates an image of blood vessel 102 using the photoacoustic effect or active ultrasound in accordance with methods known in the art. If the image indicates that blood vessel 102 is collapsed or substantially distorted, controller 62 controls piezoelectric motor 90 (FIGS. 2B, 1A, 1C) to retract pressure screw 84 and reduce pressure on region 104 so that the blood vessel reforms to substantially its natural shape. In some embodiments of the invention, if adjustment of pressure screw 84 does not result in satisfactory recovery of blood vessel 102, controller 62 generates a suitable alarm indicating that user intervention is required.

As in the case discussed above in which an anomalous glucose assay optionally initiates a realignment procedure, in some embodiments of the invention, an anomalous assay also stimulates a procedure for readjusting the position of pressure screw 84. In response to an anomalous assay, not only is the position of set screw 40 adjusted to reduce the anomaly but position of pressure screw 84 may also be adjusted to minimize the anomaly.

In some embodiments of the invention, controller 62 determines an optimum operating position for pressure screw 84. During operation in an automatic positioning mode controller 62 controls piezoelectric motor to extend pressure screw 84 to its maximum or to a point at which it senses that blood vessel 102 distorts substantially or collapses. Controller 62 then controls piezoelectric motor 90 to extract pressure screw 84 by an amount which positions the pressure screw at an operating position at which the screw is a comfortable distance from its position at which blood vessel 102 collapses or distorts. In some embodiments of the present invention, if controller 62 cannot adjust the position of pressure screw 84 so as to prevent collapse or distortion of blood vessel 102 the controller generates an alarm to alert the patient that user intervention is required.

In the above discussion of glucometer 20 specific devices, configurations and methods were described for implementing a wearable glucometer. The present invention is not limited to any of these specific devices, configurations, methods and combinations thereof and other devices, configurations, methods and combinations thereof may be used in the practice of the present invention.

For example, it is possible, in accordance with an embodiment of the invention, to provide a wearable glucometer similar in construction to glucometer 20 that does not comprise motors 78 and 90. Instead the glucometer may be configured, using any of various different devices and methods known in the art so that set screw 42 and/or pressure screw 84 may be adjusted manually. If controller 62 determines that sensor pack 24 becomes misaligned the controller generates an alarm to alert the patient that user intervention is required and that the user should adjust set screw 40 and/or pressure screw 84. It is also possible, using any of many various devices and configurations known in the art to provide manual adjustment for set screw 42 and/or pressure screw 84 for a glucometer similar to glucometer 20 in the event that motor 78 and/or 90 becomes inoperable.

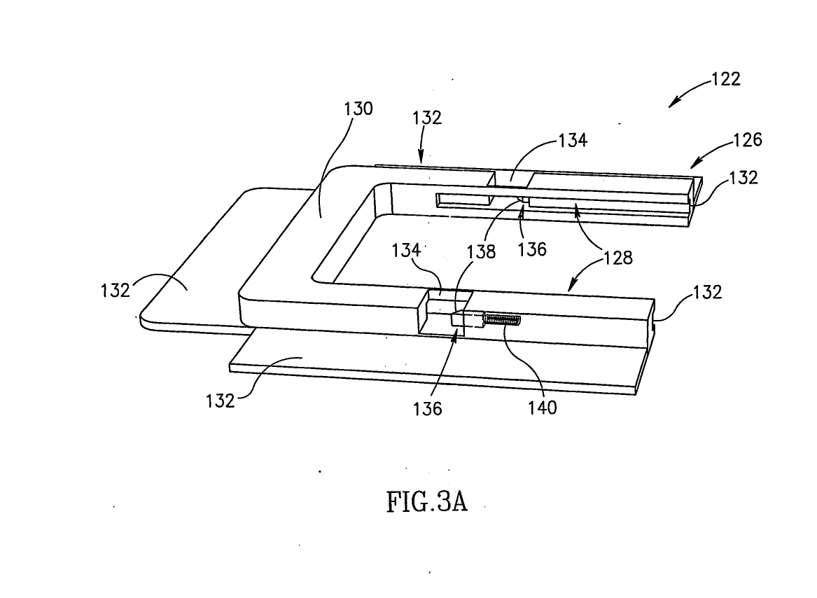

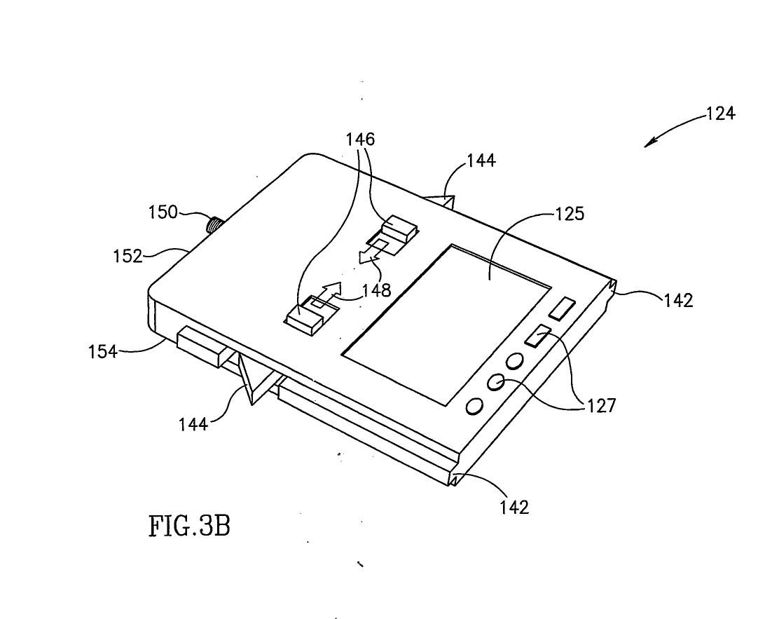

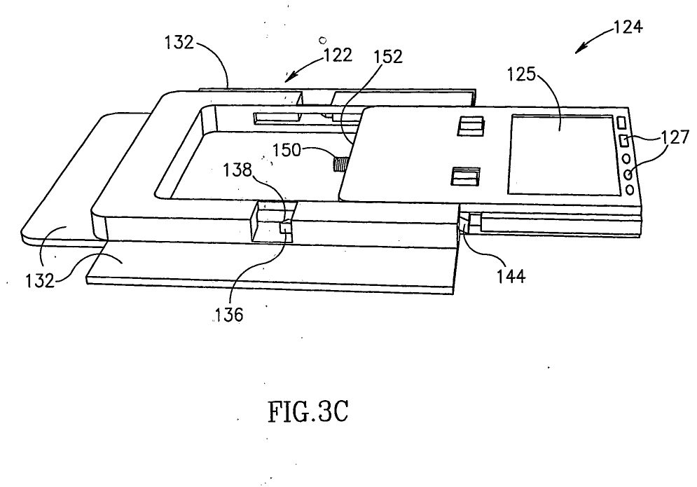

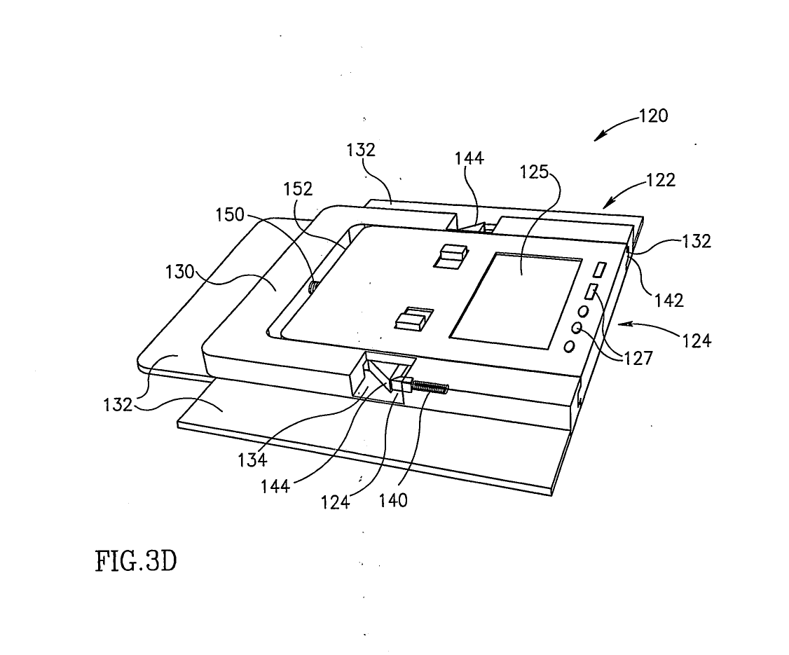

FIGS. 3A-3D schematically show respectively a mounting module 122 a sensor pack 124 and a glucometer 120 comprising the mounting module and sensor pack in accordance with yet another embodiment of the present invention.

Mounting module 122 shown in FIG. 3A comprises a U shaped frame 126 having two arms 128 and a bottom bar 130. Mounting frame is attached to flexible mounting skirts 132 having an adhesive layer for attaching the mounting module to the skin of a patient. Each arm 128 is formed with a groove 132 and a slot 134. A spring-loaded “push pin” 136 having an angled face 138 is mounted in each arm 128 using any of various techniques and devices known in the art and protrudes into each slot 134. A spring 140 that “spring-loads” push pin 136 is schematically for one of arms 128 and a portion of the push pin, which is located inside the arm in a suitable hole (not shown) formed in the arm is shown in dashed lines. Operation of push pins 136 and grooves 132 is discussed below.

FIG. 3B schematically shows sensor pack 124, which corresponds to mounting module 122. Sensor pack 124 comprises tongues 142 corresponding to grooves 132 in mounting module 122 and “catch teeth” 144 that correspond to slots 134 in the mounting module. Catch teeth 144 are spring loaded using any of various devices and techniques known in the art so that the catch teeth are resiliently pressed in directions that cause them to protrude from sensor pack 124. Release buttons 146 when pressed in directions indicated by block arrows 148 retract catch teeth 144. A set screw 150 is mounted along an edge 152 of sensor pack and is coupled to a suitable motor (not shown) inside sensor pack 124, which is controllable to increase or decrease an amount by which the sets crew protrudes from edge 152. Tongues 142, catch teeth 144 and set screw 150 cooperate as explained below in the discussion of FIGS. 3C and 3D to mount and position sensor pack 124 to mounting module 122.

Sensor pack 124 comprises at least one light source and at least one acoustic transducer (not shown) located on a bottom surface 154 of the sensor pack. As in glucometer 20, sensor pack 124 also comprises a controller and a power source (not shown) optionally mounted inside the sensor pack. When sensor pack 124 is mounted in mounting module 122 and the mounting module is attached to a patient, the light source and at least one acoustic transducer contact the patient's skin. The controller controls set screw 150, the at least one light source and at least one acoustic transducer similarly to the way in which controller 62 controls set screw 40, light source 58 and acoustic transducers 56 comprised in glucometer 20 shown in FIGS. 1A-1E. Sensor pack 122 also optionally comprises an integrated visual display 125 and control buttons 127 for inputting instructions to the controller.

Sensor pack 124 is mounted to mounting module 22 by aligning tongues 142 on the sensor pack with grooves 132 formed in the mounting module and inserting the sensor pack into the mounting module by pushing the sensor pack towards bottom bar 130 of the mounting pack. When during insertion of sensor pack 124 catch teeth 144 encounter arms 128, the force of the encounter causes the catch teeth to retract. FIG. 3C schematically shows sensor pack 124 during insertion into mounting module 22 at a point at which catch teeth 144 encounter arms 128 of the mounting module and begin to retract.

When sensor pack 124 is substantially fully inserted into mounting module 122 catch teeth 144 are located opposite their corresponding slots 134, and as a result of their being spring loaded are forced outwards into the slots. As each catch tooth 144 moves into its corresponding slot 134 it collides with angled surface 138 of push pin 136 and forces the push pin back until the push pin snaps into position behind the catch tooth and presses resiliently on the catch tooth. The position of set screw 150 determines how far sensor pack 124 is inserted into mounting module 22 and when fully inserted set screw 150 butts up against bottom bar 130 of mounting module 22. FIG. 3D schematically shows glucometer 120 fully assembled with sensor pack 124 mounted in mounting module 122 and set screw butting up against bottom bar 130 of the mounting pack.

When fully inserted into mounting module 22, push pins 136 cooperate with catch teeth 144 to resiliently press sensor pack 124 towards bottom bar 130 of the mounting module. Adjustment of the relative position of sensor pack 124 and mounting module 122 is controlled by the controller in the sensor pack which controls an amount by which set screw 150 protrudes from edge 152 of the sensor pack. The controller controls the position of the set screw responsive to alignment of sensor pack 124 with a blood vessel containing blood for which glucometer 20 is assaying glucose.

Glucometer 120 may also comprise a pressure screw and suitable motor for operating the pressure screw similar to pressure screw 84 and motor 90 comprised in glucometer 20 (FIG. 1A). For example, the light source and acoustic transducers in sensor pack 124 might be mounted in a unit that fits into a well in the bottom surface 154 (FIG. 3B) of the sensor pack. The unit is free to move up and down in the well and a pressure screw mounted in the sensor pack is operable to apply a force that tends to push the unit out of the well.

In the above described embodiments of glucometers, mounting modules (e.g. mounting modules 22 (FIG. 1A) and 122 (FIG. 3A)) comprise “open” frames (e.g. frames 30 and 126 respectively) which are attached to a person's skin, optionally with a long lasting adhesive. When a corresponding sensor pack is mounted to such a mounting module the at least one light source and the at least one acoustic transducer comprised in the sensor pack are, except for a layer of a coupling gel or adhesive, in direct contact with the skin.

In some embodiments of the invention a glucometer comprises a mounting module having a “closed” frame in which a panel, a “bottom panel”, connects the sides of the frame. The bottom panel is formed from a material that is a relatively good acoustic conductor and is substantially transparent to light provided by a light source comprised in a sensor pack used with the mounting module. The mounting module is optionally attached to a region of skin by adhering the bottom panel to the skin using an acoustically and optically transparent adhesive that reduces optical and acoustic impedance mismatch between the panel and the skin. When a sensor pack used with the mounting module is mounted to the mounting module, the sensor pack's at least one light source and at least one acoustic transducer do not contact the skin but contact instead the bottom panel.

Optionally, the sensor pack is optically and acoustically coupled to the bottom panel using a suitably optically transparent and relatively low viscosity medium, such as an appropriate gel or oil, that is also a good acoustic conductor. The viscosity of the medium should be low enough so that the sensor pack can readily be moved on the bottom panel of the mounting module to adjust the sensor pack's position relative to the module's closed frame.

A glucometer comprising a mounting module having a bottom panel, in accordance with the present invention, is optionally similar to glucometer 20 (FIG. 1A) or glucometer 120 (FIG. 3D) with the addition of a bottom panel to the glucometer's mounting module 22 and 122 respectively.

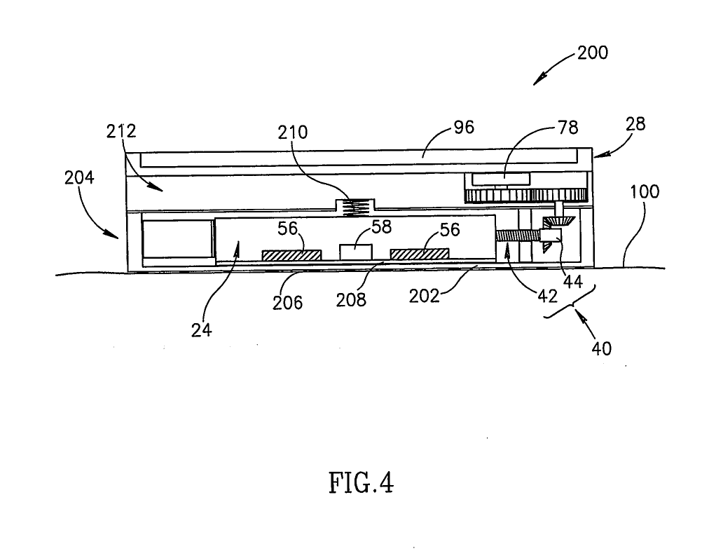

By way of example, FIG. 4 schematically shows a cross section view of a glucometer 200 comprising a bottom panel 202, in accordance with an embodiment of the present invention. Bottom panel 202 is comprised in a mounting module 204 of the glucometer. Glucometer 200 is similar to glucometer 20 (FIG. 2B) and corresponding components of glucometer 200 and glucometer 20 are labeled same numerals. Glucometer differs from glucometer 20 in that, in addition to comprising bottom panel 202, glucometer 200 optionally does not have a pressure screw 84 and associated components nor, optionally, flexible “mounting” skirts 34. Glucometer 200 is shown attached to a region of skin 100 of a patient by a layer of adhesive schematically represented by a shaded region 206. A coupling gel schematically represented by a shaded region 208 optically and acoustically couples sensor pack 24, which comprises at least one light source 58 and at least one acoustic transducer 56, to bottom panel 202. Optionally an elastic element, such as a suitable rubber disk or a leaf spring, represented by a spring 210 mounted to an actuator pack 212 resiliently presses sensor pack 24 to bottom panel 202.

A glucometer in accordance with an embodiment of the invention, such as glucometer 200, that has a bottom panel can be advantageous. The glucometer's mounting module can be stably mounted to a patient's skin for an extended period of time by using an appropriate adhesive to bond the bottom panel to the skin. The glucometer's sensor pack is relatively easily optically and acoustically coupled to the bottom panel using a suitable coupling material and the bottom panel substantially reduces loss of the coupling material due to leakage. The sensor pack may be repeatedly removed from and replaced into the mounting module without discomfort to the patient.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

1. Apparatus for assaying an analyte of blood in a patient's blood vessel comprising:
a mounting module adapted so that it can be adhered to the skin of the patient overlying a tissue region comprising the blood vessel;
a sensor unit mounted to the module that generates signals responsive to characteristics of the tissue region; and
a controller that receives the signals and uses received signals to assay the analyte and to determine a degree to which the sensor unit is aligned with the blood vessel.

2. Apparatus according to claim 1 wherein the sensor unit comprises at least one light source that illuminates the region with light at at least one wavelength that is absorbed and/or scattered by the analyte and generates photoacoustic waves in the region and at least one acoustic transducer that generates at least some of the signals responsive to the photoacoustic waves.

3. Apparatus according to claim 2 wherein the controller uses signals responsive to the photoacoustic waves to assay the analyte.

4. Apparatus according to claim 2 wherein the light source illuminates the region with light that is absorbed by red blood cells.

5. Apparatus according to claim 2 wherein the controller uses at least one characteristic of the photoacoustic signals to determine a degree to which the sensor unit is aligned with the blood vessel.

6. Apparatus according to claim 5 wherein the at least one characteristic comprises a magnitude of the amplitude of the signal.

7. Apparatus according to claim 5 wherein the at least one characteristic comprises shape of the signal.

8. Apparatus according to claim 5 wherein the at least one characteristic comprises a time dependence of the signal.

9. Apparatus according to claim 5 wherein the at least one characteristic comprises a power spectrum of the signal.

10. Apparatus according to claim 2 wherein the controller controls the at least one transducer to transmit ultrasound into the region and wherein the at least one transducer generates at least some of the signals responsive to ultrasound reflected from features comprised in the region.

11. Apparatus according to claim 10 wherein the controller uses signals generated responsive to reflected ultrasound to determine a degree to which the sensor is aligned with the blood vessel.

12. Apparatus according to claim 2 and comprising a display screen.

13. Apparatus according to claim 12 wherein the controller displays data that relates to a degree to which the sensor unit is aligned with the blood vessel on the display screen.

14. Apparatus according to claim 12 wherein the controller uses signals from the at least one acoustic transducer to generate an image of the blood vessel and displays the image on the screen.

15. Apparatus according to claim 12 wherein the controller uses signals generated responsive to reflected ultrasound to generate an image of the blood vessel and displays the image on the screen.

16. Apparatus according to claim 14 wherein the controller displays a fiducial on the screen and wherein a distance on the screen between the fiducial and the image of the blood vessel indicates a degree to which the sensor unit is misaligned with the blood vessel.

17. Apparatus according to claim 1 wherein at least a portion of the sensor unit presses against the skin to provide optical and/or acoustic coupling of the sensor to the skin.

18. Apparatus according to claim 17 wherein the controller uses signals received from the sensor unit to determine if the sensor portion exerts excessive pressure on the blood vessel.

19. Apparatus for assaying an analyte of blood in a patient's blood vessel comprising:
a mounting module adapted so that it can be adhered to the skin of the patient overlying a tissue region comprising the blood vessel;
a sensor unit mounted to the module that generates signals responsive to characteristics of the tissue region wherein at least a portion of the sensor unit presses against the skin to provide optical and/or acoustic coupling of the sensor to the skin;
a controller that receives the signals and uses received signals to assay the analyte and to determine if the sensor portion exerts excessive pressure on the blood vessel.

20. Apparatus according to claim 19 wherein the sensor unit comprises at least one light source that illuminates the region with light at at least one wavelength that generates photoacoustic waves in the region and at least one acoustic transducer that generates at least some of the signals responsive to the photoacoustic waves.

21. Apparatus according to claim 20 wherein the controller controls the at least one transducer to transmit ultrasound into the region and wherein the at least one transducer generates at least some of the signals responsive to ultrasound reflected from features comprised in the region.

22. Apparatus according to claim 18 wherein the controller uses the signals to generate an image of the blood vessel and if the image indicates that the blood vessel is deformed relative to a normative blood vessel shape, the controller determines that the sensor portion exerts excessive pressure.

23. Apparatus according to claim 18 wherein the sensor portion position is movable relative to the mounting module in a direction substantially perpendicular to the skin so as to adjust pressure that the sensor unit portion exerts on the skin and thereby on the blood vessel.

24. Apparatus according to claim 23 wherein the position of the sensor portion is manually adjustable.

25. Apparatus according to claim 23 and comprising a motor controllable to adjust the position of the sensor unit portion.

26. Apparatus according to claim 25 wherein the controller controls the motor to adjust position of the sensor unit portion if the controller determines that the sensor unit portion exerts excessive pressure on the skin.

27. Apparatus according to claim 2 wherein the mounting module comprises a frame having sides, which at least partially surround a region that receives the sensor unit.

28. Apparatus according to claim 27 wherein the region that receives the sensor unit is an open region and when the sensor unit is positioned in the region no portion of the mounting module intervenes between the sensor unit and the skin.

29. Apparatus according to claim 28 and comprising an adhesive that attaches the sensor unit to the skin when the sensor unit is mounted in the open receiving region.

30. Apparatus according to claim 29 wherein the adhesive is substantially transparent to light provided by the at least one light source.

31. Apparatus according to claim 29 wherein the adhesive is a relatively good conductor of sound and reduces acoustic impedance mismatch between the sensor unit and the skin.

32. Apparatus according to claim 28 and comprising a gel that optically and acoustically couples the sensor unit to the skin when the sensor unit is mounted in the open receiving region.

33. Apparatus according to claim 27 wherein the frame comprises a panel that connects the sides and which intervenes between the sensor unit and the skin when the sensor unit is mounted in the receiving region.

34. Apparatus according to claim 33 wherein the panel is flexible.

35. Apparatus according to claim 33 wherein the panel is substantially transparent to light provided by the at least one light source.

36. Apparatus according to claim 33 wherein the panel is a relatively good conductor of sound.

37. Apparatus according to claim 33 wherein the panel comprises an adhesive layer that bonds the panel and thereby the mounting module to the skin.

38. Apparatus according to claim 37 wherein the adhesive is substantially transparent to light provided by the at least one light source.

39. Apparatus according to claim 38 wherein the adhesive is a relatively good conductor of sound and reduces acoustic impedance mismatch between the panel and the skin.

40. Apparatus according to claim 33 and comprising a gel or oil that optically and acoustically couples the sensor unit to the panel when the sensor unit is mounted in the receiving region.

41. Apparatus according to claim 33 wherein the frame comprises at least one elastic element that exerts a resilient force on the sensor unit substantially parallel to the plane of the frame to maintain the sensor unit securely in position in the frame.

42. Apparatus according to claim 41 and comprising at least one set screw having a position that limits motion of the sensor unit in a direction that the resilient force operates to move the sensor unit.

43. Apparatus according to claim 42 wherein the set screw is mounted in the frame.

44. Apparatus according to claim 42 wherein the position of the set screw is adjustable manually.

45. Apparatus according to claim 42 and comprising a motor controllable to adjust the position of the set-screw.

46. Apparatus according to claim 45 wherein the controller controls the motor to adjust position of the sensor unit if signals received from the sensor unit indicate alignment of the sensor unit is unsatisfactory.

47. Apparatus according to claim 1 and comprising a motor controllable to adjust the position of the sensor unit relative to the mounting module in a direction parallel to the skin and wherein the controller controls the motor to adjust position of the sensor unit if signals received from the sensor unit indicate alignment of the sensor unit is unsatisfactory.

48. Apparatus according to claim 1 wherein the analyte is glucose.
